(19) United States
(12) Patent Application Publication
(10) Pub. No.: US 2024/0041318 A1
(43) Pub. Date: Feb. 8, 2024

(54) METHOD OF EVALUATING THE EFFICIENCY OF A MYOPIA CONTROL SOLUTION

(71) Applicant: Essilor International, Charenton-le-Pont (FR)

(72) Inventors: Daniel SPIEGEL, Singapore (SG); Guillaume GIRAUDET, Charenton-Le-Pont (FR); Matthieu GUILLOT, Charenton-Le-Pont (FR)

(73) Assignee: Essilor International, Charenton-le-Pont (FR)

(21) Appl. No.: 18/258,695

(22) PCT Filed: Dec. 17, 2021
(86) PCT No.: PCT/EP2021/086655

§ 371 Date: Jun. 21, 2023

(30) Foreign Application Priority Data
Dec. 23, 2020 (EP) 20306669.1

(51) Int. Cl. A61B 3/107; A61B 3/10; G06T 7/00

(52) U.S. Cl. CPC A61B 3/107; A61B 3/1005; G06T 7/0014; G06T 2207/30041

(57) ABSTRACT

A method of evaluating the efficiency of a myopia control solution for a person. The method includes providing an initial value of a prolateness indicator of at least one eye of the person, determining a second value of the prolateness indicator of the at least one eye of the person after having the person use the myopia control solution over a given period of time, and evaluating the efficiency of the myopia control solution by comparing the evolution between the initial and second values of the prolateness indicator of the at least one eye with a value of reference.

### TECHNICAL FIELD

The disclosure relates to a method of evaluating the efficiency of a myopia control solution for a person, a method for selecting at least one myopia control solution for a person among a list of myopia control solutions and a method for determining eye growth.

The disclosure further relates to a system for determining eye growth.

### BACKGROUND OF THE DISCLOSURE

Myopia of an eye is characterized by the fact that the eye focuses distant objects in front of its retina. Myopia is usually corrected using a concave lens.

Myopia, also referred as to short-sightedness, has become a major public health problem worldwide. Accordingly, a large effort has been made to develop solutions aiming to slow down myopia progression. The efficacy of these myopia control solutions is usually derived from the comparison of the average myopia progression between a reference group and a control group. Although this is informative at the population level, it does not mean that every single individual responds to the same extent to the myopia control solution.

In fact, one of the issues of myopia control solutions at the individual level is to determine to which extent an individual responds to a proposed myopia control solution. Myopia regression i.e. the eye becomes less myopic or complete stop of myopia progression are very rare. In other words, most of the individuals' refraction gets more myopic and the eye elongates even during myopia control, and the eye care professional has very little information about how much more myopic or longer the eye would have become without using any myopia control solution.

Therefore, there is a clear need to have a method that would indicate the efficiency of a myopia control solution for an individual.

### SUMMARY OF THE DISCLOSURE

To this end, the disclosure proposes a method of evaluating the efficiency of a myopia control solution for at least a person, for example a group of people, the method comprising:
providing an initial value of a prolateness indicator of at least one eye of the person,
determining a second value of said prolateness indicator of said at least one eye of the person after having the person use the myopia control solution over a given period of time,
evaluating the efficiency of the myopia control solution by comparing the evolution between the initial and second values of the prolateness indicator of said at least one eye with a value of reference.

It has been observed and confirmed that monitoring the prolateness of at least one eye of the person provides a good indication of the efficiency of a myopia control solution at an individual level.

Advantageously, the method of the disclosure allows to adapt the type of myopia control solution and/or the intensity of the myopia control solution based on the evaluated efficiency.

For example, a person responding positively to a control solution may be shifted to less aggressive form or solution. This may be beneficial for compliance as more aggressive myopia control solution can be associated with more severe side effects (e.g. reduced accommodation with Atropine, reduced visual acuity with more near adding in contact lenses or spectacles with microlenses). In addition, being able to evaluate the efficiency of a myopia control solution allows tampering down intensity of the solution and monitor any possible rebound effect.

According to further embodiments which can be considered alone or in combination:
the second value of said prolateness indicator of said at least one eye of the person is determined after a given period of time greater than or equal to 1 month, for example greater than 3 months and smaller than or equal to 36 months, for example smaller than 12 months; and/or
the prolateness indicator is determined over a given angular zone of the retina of the person; and/or
the prolateness indicator is determined at least over the nasal region of the retina of the person; and/or
the prolateness indicator is determined at least over the temporal region of the retina of the person; and/or
the prolateness indicator is determined by fitting a two-dimensional cross-section of the retina of the eye of the person with a quadratic function; and/or
the prolateness indicator is determined by fitting a two-dimensional cross-section of the retina of the eye of the person with a third-degree polynomial function; and/or
the prolateness indicator is determined based on a 3D measurement of the retina of said at least one eye of the person; and/or
the myopia control solution is selected among the list consisting of myopia control ophthalmic lenses, myopia control contact lenses, myopia control optical lenses, myopia control drugs, optical system having a specific transmission pattern; and/or
the method further comprises:
   providing an initial value of an axial length indicator of at least one eye of the person,
   determining a second value of said axial length indicator of said at least one eye of the person after having the person use the myopia control solution over the same given period of time as the prolateness indicator,
and wherein evaluating the efficiency of the myopia control solution further comprises comparing the evolution between the initial and second values of the axial length indicator of said at least one eye with a value of reference; and/or
the method further comprises
   providing an initial value of a refractive indicator of at least one eye of the person,
   determining a second value of said refractive indicator of said at least one eye of the person after having the person use the myopia control solution over the same given period of time as the prolateness indicator,
and wherein evaluating the efficiency of the myopia control solution further comprises comparing the evolution between the initial and second values of the refractive indicator of said at least one eye with a value of reference.
   The disclosure further relates to a method of evaluating the evolution of prolateness of an eye of a person, the method comprising:
providing an initial value of a prolateness indicator of at least one eye of the person,
determining a second value of said prolateness indicator of said at least one eye of the person after a given period of time,
evaluating the evolution of prolateness of an eye by comparing the evolution between the initial and second values of the prolateness indicator of said at least one eye with a value of reference.
   The disclosure further relates to a method for selecting at least one myopia control solution, for example a combination of myopia control solution, for a person among a list of myopia control solutions, comprising evaluating the efficiency of each myopia control solution for the person using a method according to the disclosure and selecting the most efficient myopia control solution.

The disclosure also relates to a method for determining eye growth, wherein the eye growth is determined by measuring over time a prolateness indicator of said eye in addition to measuring over time the axial length and/or spherical equivalent refraction of said eye.

The disclosure further relates to a system for determining eye growth, comprising at least a measuring device configured to measure and store over time an eye length indicator of an eye and a device for processing the eye length indicator of the eye over time to determine a prolateness indicator for determining eye growth.

According to a further aspect, the disclosure relates to a computer program product comprising one or more stored sequences of instructions that are accessible to a processor and which, when executed by the processor, cause the processor to carry out at least one of the steps of any of the methods according to the disclosure.

The disclosure further relates to a computer readable medium carrying one or more sequences of instructions of the computer program product according to the disclosure.

Furthermore, the disclosure relates to a program which makes a computer execute at least one of the steps of any of the methods of the disclosure.

The disclosure also relates to a computer-readable storage medium having a program recorded thereon; where the program makes the computer execute at least one of the steps of any of the methods of the disclosure.

The disclosure further relates to a device comprising a processor adapted to store one or more sequences of instructions and to carry out at least one of the steps of any of the methods according to the disclosure.

### BRIEF DESCRIPTION OF THE DRAWINGS

Non-limiting embodiments of the disclosure will now be described with reference to the accompanying drawing wherein:

FIG. 1 is a flowchart of different steps of a method for evaluating the efficiency of a myopia control solution according to the disclosure;

FIGS. 2a and 2b are schematic representations of measuring devices for measuring the axial length and peripheral length of the eye of a person;

FIG. 3 is a schematic representation of a measuring device that may be used for determining the prolateness of the eye of a person;

FIG. 4 is a flowchart of different steps of a method for selecting at least one myopia control solution for a person according to the disclosure;

FIG. 5 is a schematic representation of a system for determining eye growth according to the disclosure; and

FIG. 6 is a graph showing prolateness versus time for different myopia control solutions.

Elements in the figures are illustrated for simplicity and clarity and have not necessarily been drawn to scale. For example, the dimensions of some of the elements in the figure may be exaggerated relative to other elements to help to improve the understanding of the embodiments of the present disclosure.

### DETAILED DESCRIPTION OF EMBODIMENTS OF THE DISCLOSURE

The disclosure relates to a method of evaluating the efficiency of a myopia control solution for a person.

The myopia control solution evaluated by the method of the disclosure may be any kind of solution or combination of solutions that is to slow down progression of myopia, in particular for young kids.

For example, the myopia control solution or its combination is selected among a list consisting of: myopia control ophthalmic lenses, myopia control contact lenses, myopia control optical lenses, myopia control drugs, optical system having a specific transmission pattern.

The myopia control solution may be myopia control ophthalmic lenses comprising, in addition to a refractive zone, optical elements configured not to focus on the retina of the wearer.

The method of the disclosure is not limited to any specific myopia control solution and may be used to evaluate the efficiency, in particular over time, of any myopia control solution for a person.

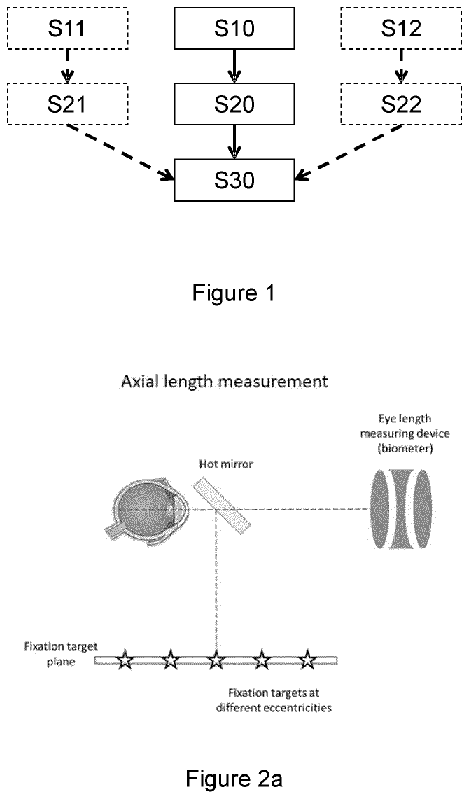

As illustrated on FIG. 1, a method according to the disclosure comprises at least:
providing an initial value of prolateness S10,
determining a second value of prolateness S20, and
evaluating the efficacy of the myopia control solution S30.

During step S10, an initial value of a prolateness indicator of at least one eye of the person is provided.

According to an embodiment of the disclosure, a prolateness indicator for both eyes of the wearer is provided in steps S10 and S20 and used in step S30.

The disclosure further relates to a method of evaluating the evolution of prolateness of an eye of a person, the method comprising:
providing an initial value of a prolateness indicator of at least one eye of the person,
determining a second value of said prolateness indicator of said at least one eye of the person after a given period of time,
evaluating the evolution of prolateness of an eye by comparing the evolution between the initial and second values of the prolateness indicator of said at least one eye with a value of reference.

In some situations it may be interesting to distinguish between the eyes of the wearer. For example, in suspected development of myopic anisometropia, i.e. one eye has substantially more myopia than the other, one may want to monitor its progression and even compare the difference of prolateness between both eyes over time.

The opposite may also be possible, one may want to monitor that the less myopic eye is not trying to catch up with the more myopic one. A focus on the dominant eye of the wearer may be advantageous in some specific situations, for example when one eye is blind or severely impaired.

The prolateness of the eye may be characterized by determining the shape of the retina of the eye of the person at least over a given angular zone of the retina of the person. The given angular zone is of at least 5° on the nasal part, for example at least 10° on the nasal part, preferably at least 15° on the nasal part, and of at least 5° on the temporal part, for example at least 10° on the temporal part, preferably at least 15° on the temporal part.

According to an embodiment of the disclosure, the prolateness indicator is determined at least over the nasal region of the retina of the person. Indeed, it has been observed that over a short period of time, typically greater than 1 month and smaller than 9 months, the prolateness of the eye in the nasal region of the retina is more discriminating relative to the efficiency of the myopia control solution.

According to an embodiment of the disclosure, the prolateness indicator is determined at least over the temporal region of the retina of the person. Indeed, it has been observed that over a long period of time, typically greater than 10 months and smaller than 36 months, the prolateness of the eye in the temporal region of the retina is more discriminating relative to the efficiency of the myopia control solution.

The data about the ocular shape can be obtained with different measurement methods such as:
(Relative) peripheral refraction—for example 30° deg temporal, 15° deg temporal, fovea, 30° deg nasal, 15° deg nasal
(Relative) peripheral axial length—for example 30° deg temporal, 15° deg temporal, fovea, 30° deg nasal, 15° deg nasal
optical coherence tomography or OCT—for example 6 mm b-scan
Magnetic resonance imaging or MRI

Although there may not be any available device for measuring peripheral eye length as such, getting these data is relatively simple. Several possible ways that have varying levels of complexity are described here. All may be implementable into a standalone device.

Most of the setups/devices described below allow for measurement at distinct retinal eccentricities or as a continuum.

A first approach was described in Ding, X., & He, M. (2012). Measurement of Peripheral Eye Length. Ophthalmology. This method utilizes the standard device (IOLMaster) used for axial eye length measurement and is equipped with peripheral targets affixed onto the sides of the lateral aperture of IOLMaster. These targets correspond to a specific retinal/visual field eccentricity. The person fixates the target whilst the measurement is taken.

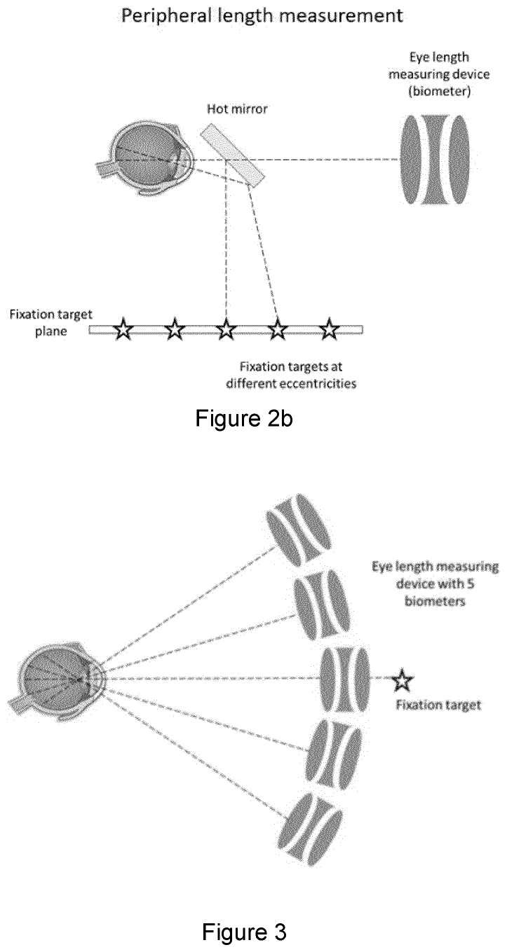

A second approach is to use a standard axial eye length device (e.g. IOLMaster, LenStar, etc.) together with an infrared hot mirror allowing the eye length beam to pass and to see off-axis fixation targets. Such setup is illustrated on FIGS. 2a and 2b and may be embedded in a standalone device, i.e., the plane the fixation target plane with the eccentric targets would be inside of the device.

A third approach is to have a device with multiple eye length measuring beams as illustrated on FIG. 3. A similar device BHVI-EyeMapper exists. The main difference is that the BHVI-EyeMapper measures refraction and aberration but not eye length. An alternative may be a device with one eye length measuring beams rotating in front of the eye.

The data collected may be subjected to the data analysis described hereafter to determine a prolateness indicator of the eye of the person.

A typical way of acquisition of the ocular shape data using axial length or refraction is done according to naso-temporal b-scan (two-dimensional cross-section).

Mathematically, the prolateness can be for example quantified by fitting the posterior eye shape data by a quadratic function.

The quadratic function may be for example, f(x)=−a*(x+b)2+c
where “x” is visual field angle, “a” quantifies the prolateness, “b” factor specifies the x position (i.e., retinal position) of the peak of the function, and “c” is the y position (i.e. best fitting central axial length).

For example, a specific procedure for obtaining the prolateness is to find such a, b, and c parameters to minimize the sum of squared residual, i.e., the difference between an observed value, and the fitted value provided by the quadratic function, commonly referred to as least squares analysis, over a number of iterations, for example 1000. The best fitting a, b, and c parameters, i.e., the sum of least squared residuals across the iterations is the smallest, are considered best representatives of the retinal shape and the term “a” is taken as an indicator of the prolateness of the eye.

According to an embodiment of the disclosure, the prolateness indicator is determined by fitting a two-dimensional cross-section of the retina of the eye of the person with a third-degree polynomial function.

For example, the prolateness indicator is determined using the first derivative of the best fitting third polynomial function (f(x)=ax³−bx²+cx+d) to the retinal data.

Specifically, one can minimize the sum of squared residuals, i.e., the difference between an observed value, and the fitted value provided by the third polynomial function, over a number of iterations by varying the parameters “a”, “b”, “c”, and “d”.

The best fit is then subjected to the first derivative and the mean, for example the absolute value to account for opposite signs on the sides of the fovea, of the first derivative provides an indication of the prolateness.

An advantage of this approach is that the asymmetrical third-degree polynomial function usually provides a better fit to the retinal data compared to a strictly symmetrical function.

Secondly this approach allows choosing only a certain region of the retina over which the mean of the derivative is calculated, i.e., prolateness. In its most localized version, it allows to quantify retinal steepness at one particular point. Another way to quantify the retinal steepness at a given point is to find the tangent of the polynomial function at that point and determine its angle.

Furthermore, this approach allows quantifying the retinal asymmetry, i.e. the difference between the prolateness/steepness of nasal and temporal retina.

According to an embodiment of the disclosure, the prolateness indicator may be determined based on a 3D measurement of the retina of said at least one eye of the person.

The ocular shape data can be acquired at different locations or orientations across the retina for example by denser sampling or using an advanced imaging modality such as optical coherence tomography or OCT or MRI system. In case of such continuous retinal shape data, the retinal shape parameters can be calculated by the methods described previously. Furthermore, imaging techniques such as optical coherence tomography allow for volume data acquisition that allow for calculation of retinal prolateness maps.

A similar approach to mapping prolateness, can be used for choroidal thickness, i.e. mapping choroidal thickness changes in a volume.

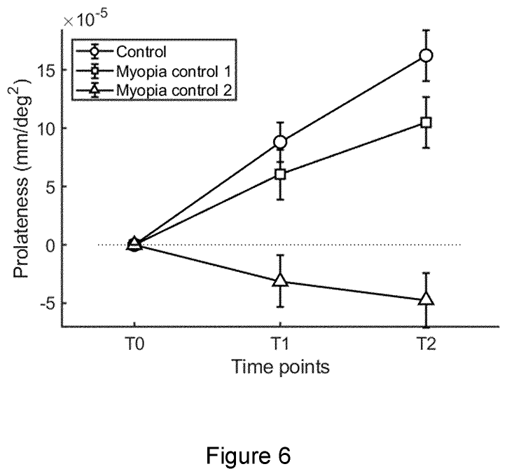

The prolateness appears to be a good indicator of the efficiency of a myopia control solution. A first group of people using a first myopia control solution, a second group of people using a second myopia control solution and a control group of people without any myopia control solution have been compared as illustrated in FIG. 6.

It has been observed that while most of the eyes in the different groups increased their axial length and their myopic refraction, the prolateness of the eyes in the first and second groups, using myopia control solutions, extended less than the control group or even decreased. Therefore, prolateness emerges as a new indicator of the efficiency of myopia control solution.

An example, for children with myopia between −0.75 D and −4.75 D, the average annual changes in prolateness without myopia control is 1.7×10⁻⁴ mm/deg² and 0.9×10⁻⁴ mm/deg² (Myopia control 1) and −0.4×10⁻⁴ mm/deg² (myopia control 2) with two different myopia control solutions.

The average annual changes in prolateness, with or without myopia control, may be different across ethnicities because different ethnicity groups with the same refractive error range differ in prolateness levels. Similarly, average annual changes in prolateness, with or without myopia control, may be different between genders because genders of the same refractive group differ in prolateness levels.

The simple quantification of naso-temporal prolateness can serve as an indicator of myopia control solution efficiency. Ideally, the eye does not become more prolate or even become less prolate despite axial elongation and/or increase in myopic refraction to determine the efficiency of the myopia control solution for the person.

To get an idea about the person's response to myopia control solution, other factors can be taken into account. For example, axial length and/or refraction.

As illustrated on FIG. 1, the method of the disclosure may further comprise:
providing S11 an initial value of an axial length indicator of at least one eye of the person, and
determining S21 a second value of said axial length indicator of said at least one eye of the person after having the person use the myopia control solution over the same given period of time as the prolateness indicator.

The evaluation of the efficiency of the myopia control solution may further comprises comparing the evolution between the initial and second values of the axial length indicator of said at least one eye with a value of reference.

As illustrated on FIG. 1, the method of the disclosure may further comprise:
providing S12 an initial value of a refractive indicator of at least one eye of the person,
determining S22 a second value of said refractive indicator of said at least one eye of the person after having the person use the myopia control solution over the same given period of time as the prolateness indicator.

The evaluation of the efficiency of the myopia control solution may further comprises comparing the evolution between the initial and second values of the refractive indicator r of said at least one eye with a value of reference.

Advantageously, this allows obtaining a more complete picture about the efficiency of the myopia control solution for the person. For example, one may consider the change of prolateness and change in refraction and can categorize the person into four categories:
Progressing responder: more myopia, decrease or no change in prolateness
Progressing non-responder: more myopia, increase in prolateness
Regressing responder: less myopia, decrease or no change in prolateness
Regressing non-responder: less myopia, increase in prolateness

Each category can be dealt with differently. For example, whilst in regressing responder, the practitioner can be fairly certain about the response to the myopia control solution, in progressing non-responder, alternative myopia control solutions should be considered.

The disclosure also relates to a method for selecting at least one myopia control solution for a person among a list of myopia control solutions.

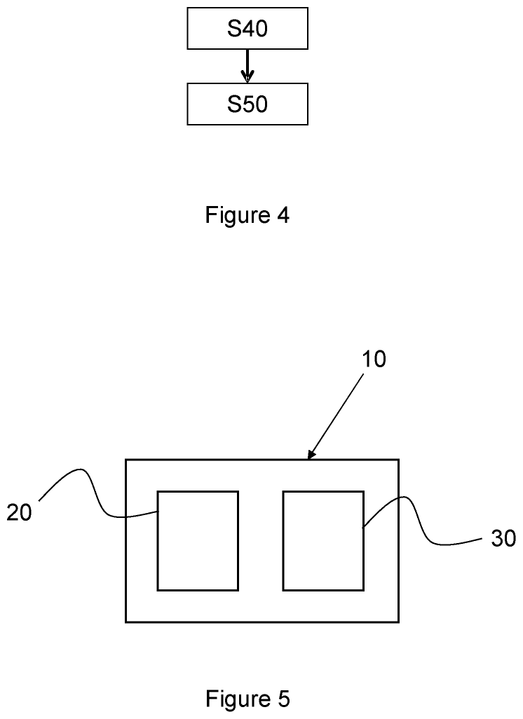

As illustrated on FIG. 4, such method may comprise evaluating S40 the efficiency of each myopia control solution for the person using a method according to the disclosure and selecting S50 the most efficient myopia control solution for the person.

Advantageously, the method of the disclosure allows providing the most adapted myopia control solution to the person based on a measurable parameter and eventually adapt or change the myopia control solution of the person based on the measured prolateness.

The disclosure further relates to a method for determining eye growth of a person. The eye growth is determined by measuring over time a prolateness indicator of said eye in addition to measuring over time the axial length and/or spherical equivalent refraction of said eye.

The method of the disclosure may also be for monitoring the eye growth of a person over time by repeating the prolateness measurements over time.

The disclosure may also relate to a method for predicting the eye growth of a person based on prolateness indicator of the eye of the person.

As described previously, the disclosure also relates to a system 10 for determining eye growth of a person.

As illustrated on FIG. 5, the system 10 comprises at least a measuring device 20 configured to measure and store over time an eye length indicator of an eye and a device 30 for processing the eye length indicator of the eye over time to determine a prolateness indicator for determining eye growth.

The measuring device may be any of the measuring device described in reference to FIGS. 2a to 3.

Many further modifications and variations will be apparent to those skilled in the art upon making reference to the foregoing illustrative embodiments, which are given by way of example only and which are not intended to limit the scope of the disclosure, that being determined solely by the appended claims.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the disclosure.

1. A method of evaluating the efficiency of a myopia control solution for a person, the method comprising:
providing an initial value of a prolateness indicator of at least one eye of the person;
determining a second value of said prolateness indicator of said at least one eye of the person after having the person use the myopia control solution over a given period of time; and
evaluating the efficiency of the myopia control solution by comparing the evolution between the initial and second values of the prolateness indicator of said at least one eye with a value of reference.

2. The method according to claim 1, wherein the prolateness indicator is determined over a given angular zone of the retina of the person.

3. The method according to claim 1, wherein the prolateness indicator is determined at least over the nasal region of the retina of the person.

4. The method according to claim 1, wherein the prolateness indicator is determined at least over the temporal region of the retina of the person.

5. The method according to claim 1, wherein the prolateness indicator is determined by fitting a two-dimensional cross-section of the retina of the eye of the person with a quadratic function.

6. The method according to claim 1, wherein the prolateness indicator is determined by fitting a two-dimensional cross-section of the retina of the eye of the person with a third-degree polynomial function.

7. The method according to claim 1, wherein the prolateness indicator is determined based on a 3D measurement of the retina of said at least one eye of the person.

8. The method according to claim 1, wherein the myopia control solution is selected among the list consisting of: myopia control ophthalmic lenses, myopia control contact lenses, myopia control optical lenses, myopia control drugs, optical system having a specific transmission pattern.

9. The method according to claim 1, further comprising:
providing an initial value of an axial length indicator of at least one eye of the person; and
determining a second value of said axial length indicator of said at least one eye of the person after having the person use the myopia control solution over the same given period of time as the prolateness indicator, and wherein evaluating the efficiency of the myopia control solution further comprises comparing the evolution between the initial and second values of the axial length indicator of said at least one eye with a value of reference.

10. The method according to claim 1, further comprising:
providing an initial value of a refractive indicator of at least one eye of the person; and
determining a second value of said refractive indicator of said at least one eye of the person after having the person use the myopia control solution over the same given period of time as the prolateness indicator, and wherein evaluating the efficiency of the myopia control solution further comprises comparing the evolution between the initial and second values of the refractive indicator of said at least one eye with a value of reference.

11. A method for selecting at least one myopia control solution for a person among a list of myopia control solutions, comprising evaluating the efficiency of each myopia control solution for the person using the method according to claim 1, and selecting the most efficient myopia control solution.

12. A method for determining eye growth,
wherein the eye growth is determined by measuring over time a prolateness indicator of said eye in addition to measuring over time the axial length and/or spherical equivalent refraction of said eye.

13. A system for determining eye growth, comprising at least a measuring device configured to measure and store over time an eye length indicator of an eye and a device for processing the eye length indicator of the eye over time to determine a prolateness indicator for determining eye growth.

14. A computer program product comprising one or more stored sequences of instructions that are accessible to a processor and which, when executed by the processor, causes the processor to carry out the steps of claim 1.

15. A computer readable medium carrying one or more sequences of instructions of the computer program product of claim 14.
